(19) United States
(12) Patent Application Publication
(10) Pub. No.: US 2009/0047315 A1
(43) Pub. Date: Feb. 19, 2009

(54) POROUS MAGNESIA AND PROCESS FOR PREPARING THE SAME

(76) Inventors: Tamio Noguchi, Fukushima Ken (JP); Yukitaka Watanabe, Fukushima Ken (JP); Fumiko Sasaki, Fukushima Ken (JP)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/279,356

(22) PCT Filed: Jan. 17, 2007
(86) PCT No.: PCT/EP2007/000351

§ 371 Date: Aug. 14, 2008

(30) Foreign Application Priority Data
Feb. 14, 2006 (JP) 2006-036848

(51) Int. Cl. A61K 8/02; C09D 7/12; A01N 25/00; A61K 9/00; A61L 9/01; A61L 9/012; C08K 3/00; C08K 3/22; A61P 25/24

(52) U.S. Cl. 424/401; 106/311; 424/409; 502/439; 424/484; 424/63; 424/76.1; 524/436

(57) ABSTRACT

The present invention relates to porous magnesia of substantially spherical shape having a silica layer, which may be used as a carrier for deodorants, antibacterials, catalysts, slow action agents (the adsorbent having the possibility of decreasing the effect of the co-administered medicines or fertilizer and plastic additives, and body pigments.

### TECHNICAL FIELD

The present invention relates to porous magnesia of substantially spherical shape having a silica layer, which may be used as a carrier for deodorants, antibacterials, catalysts, slow action agents (the adsorbent having the possibility of decreasing the effect of the co-administered medicines or fertilizer and plastic additives, and body pigments.

### BACKGROUND ART

As the porous material, porous magnesia (patent document 1) and basic magnesium carbonate (patent document 2) are known. Porous magnesia has its uses in sintered bodies suitable for furnace materials and sheath materials, and basic magnesium carbonate has its uses in various fillers, filling materials for rubbers, carriers for agricultural medicaments and catalysts, cosmetics, and others.

In recent years, due to intense heat partly caused by the effects of global warming, there has been an increasing need for deodorant products for deodorizing uncomfortable sweat odor, thus the use of porous materials. The malodor components which cause such uncomfortable sweat odor are classified into three major types: lower aliphatic acids, amines, and vinyl ketones which are aging odor generated through the oxidation of unsaturated aliphatic acids (non-patent document 1). Although conventional deodorant products have some deodorizing effects individually on each malodor of lower aliphatic acids, amines, and vinyl ketones in aging odor, there are currently few deodorants for effectively deodorizing such all malodor components.

Although inorganic compounds such as fine particulate magnesium oxide and zinc oxide are being used especially due to their high deodorizing speeds and efficiencies (non-patent document 2), these deodorants have a problem of poor dispersion during their preparation processes as well as a problem of having a poor feel during use (non-patent document 3).

Although there are known inorganic compounds for chemically deodorizing lower aliphatic acids (propionic acid, butyric acid, caproic acid, isovaleric acid), such as apatite hydroxide, hybrid powder in which fine particles of zinc oxide are carried by nylon powder, and aluminosilicate-base deodorants, these have problems in that their deodorizing speeds and efficiencies are insufficient.

Also for deodorizing vinyl ketone odors which are known as the aging odor, there are known deodorants with good deodorizing efficiency, such as amorphous alumina-silica, laminar silicate compounds, and spherical porous silica coated with magnesia (patent documents 3 to 6); however, these have insufficient efficiencies in deodorizing isovaleric acid and amines which are the principal component of body odors components (non-patent document 4), among these especially foot odor and axillary odor.

There has been report of magnesium oxide and silica (oxides of magnesium and silicon elements) as deodorant, ones which are complexed using silicon dioxide and magnesium oxide as the principal raw materials (patent documents 7 and 8) and ones which are a mixture of magnesium oxide and aluminosilicate (patent document 6). Patent document 6 describes a mixture of magnesium oxide and aluminosilicate to be used as a deodorant, and patent documents 7 and 8 describe a deodorant in which the mass ratio of silicon dioxide/magnesium oxide is preferably 1 to 14 and the mass content of silicon dioxide is not less than 50 wt %.

In the above mentioned chemical deodorization methods, no substance has been found which can efficiently deodorize malodor substances such as acidic lower aliphatic acids constituting the body odor, vinyl ketone components constituting the aging odor, and basic amines. Also, these known arts did not provide a satisfactory feel of use when applied onto human body.

[Patent document 1] JP, A, 04-338179
[Patent document 2] JP, A, 63-89418
[Patent document 3] JP, A, 07-138140
[Patent document 4] JP, A, 10-338621
[Patent document 5] JP, A, 2002-68949
[Patent document 6] JP, A, 2001-187721
[Patent document 7] JP, A, 2003-73249
[Patent document 8] JP, A, 2004-168668
[Non-patent document 1] J. Soc. Cosmet. Chem. Japan. 37(3) 195-201
[Non-patent document 2] J. Soc. Cosmet. Chem. Japan. Vol. 29., No. 1., p55-63, 1995
[Non-patent document 3] J. 1., Soc. 1., Cosmet. Japan., Vol 23(3), P217-224, 1989
[Non-patent document 4] J. Soc. Cosmet. Chem. Japan. 37(3) P202-209 (2003)

### DISCLOSURE OF THE INVENTION

### Problems to be Solved by the Invention

Accordingly, it is an object of the present invention to provide a carrier used in the application areas in which a porous structure is required, especially porous magnesia, further comprising a silica layer which efficiently deodorizes body odors such as axillary odor, sweat odor and foot odor, including especially uncomfortable aging odors and further provides a good feel of use, and deodorants and deodorant cosmetics containing the aforementioned substances.

### Means for Solving the Problems

After having conducted an earnest investigation to solve the above described problems, surprisingly the inventors have found that porous magnesia, which comprises substantially spherical particles forming a base of the porous magnesia and having a structure in which thin platelets of a magnesium compound are combined and/or intersected in two or more different directions, and a hydrated silicon oxide layer coated on the base, provides a porous structure with a large surface area occupied by mesopores and can suitably be used in the application areas in which a porous structure is required, especially in deodorants.

Accordingly, the present invention relates to porous magnesia of substantially spherical shape comprising substantially spherical particles forming a base of the porous magnesia and having a structure in which thin platelets of a magnesium compound are combined and/or intersected in two or more directions, and wherein hydrated silicon oxide is forming an outer layer of the particles.

Moreover, the present invention relates to the above described porous magnesia further comprising a magnesium compound layer as the outer most layer thereof.

Further, the present invention relates to the above described porous magnesium, wherein the amount of hydrated silicon oxide is 5 to 50 wt % as SiO₂ with respect to the total weight the porous magnesia.

Further, the present invention relates to the above described porous magnesium, wherein the magnesium compound is one or more kinds selected from the group consisting of hydrated oxide, basic carbonate, and oxide of magnesium, and the hydrated silicon oxide is hydrated silicon oxide and/or silica.

Further, the present invention relates to the above described porous magnesia, wherein the magnesium compound is a complex metal hydroxide, a complex metal carbonate, and/or a complex metal oxide between magnesium and one or more kinds of metal components selected from the group consisting of aluminum, zinc, and iron.

Further, the present invention relates to the above described porous magnesium, wherein the atomic ratio of the other metal component to magnesium: M/Mg (where M is any of Al, Z or Fe, or a mixture thereof), is not more than 0.95.

Further, the present invention relates to the above described porous magnesia, wherein the mean particle diameter of the porous magnesia is 5 to 50 μm.

Further, the present invention relates to the above described porous magnesia, wherein the proportion of the specific surface area of the mesopores having a pore diameter of 2 to 50 nm is not less than 80% with respect to the total specific surface area of the porous magnesia.

Furthermore, the present invention relates to the above described porous magnesium, wherein the oil absorption thereof is 300 to 600 ml/100 g. Further, the present invention relates to the above described magnesium, wherein the friction coefficient thereof measured by a KES friction tester is not more than 0.6.

Further, the present invention relates to a preparation process of the above described porous material, comprising the steps of: into water simultaneously adding dropwise
(A-1) a single aqueous solution of magnesium metal salt or a mixed aqueous solution of magnesium metal salt and other metal salt, and
(B-1) an aqueous alkaline solution or an aqueous carbonate solution to obtain substantially spherical particles having a structure in which thin platelets composed of hydrated metal oxide and/or carbonate of those metals are combined and/or intersected in two or more different directions;
coating the surfaces of the above described particles with hydrated silicon oxide from
(B-2) an aqueous alkali metal silicate solution, and
(A-2) a dilute aqueous mineral acid solution; and separating, washing, drying and, if desired, calcining the resultant suspension.

Further, the present invention relates to the above described preparation process, comprising the steps of: onto the outer layer of porous magnesia, further simultaneously adding dropwise
(A-3) a single aqueous solution of magnesium metal salt or a mixed aqueous solution of magnesium metal salt with other metal salt, and
(B-3) an aqueous alkaline solution or an aqueous carbonate solution to coat a hydrated oxide and/or carbonate of those metals; and
separating, washing, drying and, if desired, calcining the resultant suspension.

Further, the present invention relates to the above described process, wherein the single aqueous solution of magnesium metal salt or the mixed aqueous solution of magnesium metal salt and other metal salt includes sulfate ions, and the ion concentration ratio of sulfate ion/magnesium ion, or the ion concentration ratio of sulfate ion/magnesium ion plus other metal ion is 0.3 to 2.0.

Further the present invention relates to the use of the above described porous magnesia as a carrier for an antibacterial, a catalyst, an antidepressant or a plastic additive, or a body pigment.

Further, the present invention relates to the use of the above described porous magnesia as a deodorant.

Further, the present invention relates to a deodorant containing the above described porous magnesia.

Further, the present invention relates to deodorant cosmetics containing the above described porous magnesia.

The inventors have reported that a particle having a structure in which thin platelets of magnesium compound are combined and/or intersected in two or more different directions can take a substantially spherical shape and has a good disintegrability, it has a good sliding property, adhesiveness, and an oil absorbing property as a cosmetic body pigment (see JP, A, 2003-261796). The present invention is originated from the porous material having this unique structure.

### ADVANTAGES OF THE INVENTION

Porous magnesia of the present invention has a very strong deodorizing effect, and is effective in deodorizing malodors consisting of alkali odors such as ammonia, amines, and pyridine; acidic odors of lower aliphatic acids such as isovaleric acid; vinyl ketone of aging odors, as well as neutral odors such as esters and aldehydes. Moreover, since the porous magnesia of the present invention can take a substantially spherical shape and is easily disintegrable, it has a good sliding property, adhesiveness, oil absorption property, and excellent usability for skin, and therefore is suitable for use in cosmetics.

While HP-MS (highly porous magnesia/silica) powder disclosed in patent documents 3 and 4 as well as in non-patent document 4 was unsatisfactory especially in terms of the deodorization rate of isovaleric acid and methylamine, the porous magnesia of the present invention exhibits excellent deodorization rates not only for vinyl ketones but also for isovaleric acid and methylamine.

Moreover, since the porous magnesia of the present invention has a porous structure having a large surface area, it is suitable for use in the application areas in which such feature is required, such as carriers for antibacterials, catalysts, antidepressant and plastic additives, and body pigments.

### BEST MODE FOR CARRYING OUT THE INVENTION

Hereinafter, the porous magnesia of substantially spherical shape according to the present invention will be described in more detail along with the preparing process of the same. The substantially spherical particles which provide the base of the porous magnesia of substantially spherical shape according to the present invention can be prepared based on the disclosure of JP, A, 2003-261796. A precipitation method is adopted, which uses (1) an aqueous alkaline solution when hydrated magnesium oxide is used for the magnesium compound for the porous magnesia of substantially spherical shape according to the present invention, or (2) an aqueous carbonate solution when basic magnesium carbonate is used for it. Further, when magnesium oxide (magnesia) is used as magnesium compound in the present invention, the porous magnesia of substantially spherical shape can be prepared through the steps of: coating a hydrated silicon oxide layer onto hydrated magnesium oxide or basic magnesium carbonate in the form of the above described substantially spherical particles; if desired, coating a magnesium compound thereon; and thereafter calcining the substantially spherical particles obtained from the suspension.

Hereinafter, the preparation process of the substantially spherical particles which provide the base of the porous magnesia of substantially spherical shape having a silica layer according to the present invention will be described in more detail. By using an aqueous solution of magnesium salt compound and an aqueous alkaline solution or an aqueous carbonate solution, and adopting simultaneous dropwise addition thereof, it is possible to obtain substantially spherical particles consisting of hydrated magnesium oxide or magnesium carbonate. During this process, when complexed with other metal salt, substantially spherical particles can be obtained by use of an aqueous magnesium salt solution and an aqueous solution of the other metal salt. The magnesium salt compound used in the preparation process includes magnesium sulfate, magnesium nitrate, magnesium chloride, magnesium acetate, and magnesium oxalate.

Other metals used for complexing the magnesium compound with other metallic compound in the present invention include aluminum, zinc, and iron; those various salts thereof can be used in an aqueous solution together with magnesium salt. The ratio of magnesium compound to other metallic compound to be complexed: M/Mg (where M is any of Al, Zn or Fe, or a mixture thereof is preferably not more than 0.95, and more preferably not more than 0.7. When it is not less than 0.95, substantially spherical particles will not be produced, which will consequently reduce the feel of use (friction and extendibility) and therefore is undesirable.

As other metal salts, aluminum potassium sulfate, aluminum chloride, and aluminum sulfate, etc. are adopted for aluminum; zinc sulfate, zinc chloride, and zinc nitrate, etc. for zinc; and iron chloride, iron sulfate, ferrous sulfate, ferric sulfate and ferric nitrate, ferric ammonium alum, etc. for iron, and use of water soluble metal salts are recommended. Although use of water soluble salts is preferable, it is sufficient if they are water soluble under a heated condition for reaction. Although the aqueous solution of magnesium salt, or metal salt consisting of magnesium salt and other metal salt, which is to be prepared in advance in the present invention, may have any concentration in principle, provided that the salt is dissolved completely, typical concentrations of 0.2 to 1.0 mol/liter are adopted.

Alkali components used for hydrolysis in the present invention include sodium hydroxide, potassium hydroxide, and ammonium hydroxide. On the other hand, when carbonate is adopted, a carbonate compound is used in an aqueous solution in place of the afore-mentioned alkali components. As the carbonate compound, sodium carbonate, potassium carbonate, or ammonium carbonate will be adopted. In preparing the substantially spherical particle base of the invention, it is likely to be easier to obtain porous and substantially spherical particles, which is the final objective property, when adopting a basic carbonate form than when adopting a hydrated oxide form as an intermediate product. Furthermore, in preparing substantially spherical particles of the base of the invention, it becomes easier to obtain spherical particles of a porous structure, which is the objective property, by configuring the ion concentration ratio of sulfate ion to that of metal salt ion of magnesium compound (including also the cases complexed with other metal salts) to be 0.3 to 2.0, as described in JP, A, 2003-261796, even when processing via hydrated oxide as an intermediate product.

The preparation process of the substantially spherical particles, which provide the base, adopted in the present invention comprises separately preparing an aqueous solution of magnesium salt (and other metal salt when complexed with it) and either an aqueous alkaline solution or an aqueous carbonate solution, and simultaneously adding dropwise the aqueous solutions to hot water, which has been separately heated, while stirring and keeping the pH constant in the range of 7.5 to 11, preferably 8.0 to 10.5. In this process, the hot water before the dropwise addition preferably has a regulated sulfate ion concentration especially when the dropwise addition is performed with an aqueous alkaline solution as described above. The reaction temperature adopted in the present invention is preferably not lower than 50° C., preferably in the range of 70° C. to 90° C. in terms of the ease of formation of spherical particles.

In the step of the dropwise addition, it is necessary to simultaneously add an aqueous solution of metal salt and an aqueous alkaline solution or an aqueous carbonate solution. When the addition is not performed simultaneously, or the addition is performed without regulating the pH, it is likely that the resultant particles have an off-spherical shape and a non-uniform size, which is undesirable.

Next, the coating step of hydrated silicon oxide will be described below. Layer of hydrated silicon dioxide according to the present invention preferably means particles of hydrated silicon dioxide deposited onto the surface of the base material, resulting in a more or less dense layer. For proceeding to the coating step of hydrated silicon oxide, the suspension of substantially spherical particle base obtained as described above can be provided to the coating step of hydrated silicon oxide as it is, or after concentrating it with the substantially spherical particles through the operations of sedimentation (removing the supernatant fluid after settling), filtration, or centrifugal separation. Further, it is also possible to proceed to the coating step of hydrated silicon oxide by obtaining a suspension again through a filtering and drying step. By using the substantially spherical particle base obtained through such concentration and filtration, it is possible to downsize the volume of the reaction chamber for the subsequent coating step of hydrated silicon oxide, which is preferable in terms of production efficiency. Thus, the substantially spherical particle base is adapted to have a predetermined suspension (slurry) concentration.

Next, an aqueous solution of alkali metal silicate compound and dilute mineral acid are simultaneously added dropwise to the above described slurry under heating and stirring while keeping the pH constant in the range of 6.5 to 10.0, preferably 7.0 to 9.0. Thus, it is possible to coat hydrated silicon oxide particles uniformly onto the substantially spherical particle base. The alkali metal silicate compound used in the present invention includes sodium silicate and potassium silicate. As mineral acids for precipitating hydrated silicon oxide from a silicate compound, a diluent of hydrochloric acid, nitric acid, or sulfuric acid is used.

Further, if desired, after hydrated silicon oxide is coated, a magnesium compound is further coated onto the outer layer in a manner similar to the above described preparation process of substantially spherical particle base. In this magnesium compound layer, when complexing (doping) magnesium with other metal salt (other metallic components consisting of one or more kinds selected from the group consisting of aluminum, zinc, and/or iron), the atomic ratio of the other metal to magnesium: M/Mg (where M is any of Al, Zn or Fe, or a mixture thereof in the final form of porous magnesia of substantially spherical shape having a silica layer may be not more than 0.95, preferably not more than 0.7, although it may be different from the composition of the substantially spherical particle base.

Consequently, as the final form, the amount of hydrated silicon oxide layer may be 5 to 50 wt %, preferably 10 to 30 wt % as silica (SiO₂). With contents lower than 5 wt %, the deodorizing efficiency for amines is likely to be reduced and when, on the contrary, with contents higher than 50 wt %, the deodorizing effect against acidic matters is likely to be reduced.

The resultant precipitate is filtered/recovered by a filter or centrifugal separator, and washed and dried. By appropriately selecting the temperature and the drying time of the drying step, the precipitate may be obtained in a state of hydrated oxide or a state of carbonate; further when, as desired, obtaining the precipitate as oxide, a process of calcining at a temperature higher than the drying temperature is adopted.

Drying may be performed at a temperature of 105° C. to 150° C. and when obtaining oxides from hydroxide or carbonate through a calcining process after further drying, a temperature of not lower than 400° C., preferably 400° C. to 800° C. is adopted.

Moreover, in the present invention, a silica layer refers to, after a drying step, a layer of a single or mixed state of hydrated silicon oxide and/or silica as oxide, and further to a layer of silica in the form of oxide obtained by calcining.

The size of the porous magnesia of substantially spherical shape having a silica layer obtained according to the present invention is chosen to be in the range of 5 to 50 μm, preferably 10 to 25 μm to provide a good sliding property in consideration of abnormal feel during usage, etc. Especially, this range of particle size provides a good feet to the skin. Larger sizes than this range will reduce the adhesiveness to the skin, and smaller sizes will reduce the extensibility. The measurement of the diameter of the afore-mentioned particles can be performed by use of various particle size measuring instruments. Specifically, the measurement can be performed using Mastersizer-2000 (from Malvern) based on the laser scattering method.

The surface of the porous magnesia of substantially spherical shape obtained according to the present invention has pores composed of mesopores (diameter: 2 to 50 nm) and micropores (diameter: not larger than 2 nm). And, the proportion of the specific surface area made up of mesopores (diameter: 2 to 50 nm) is not less than 80%, preferably not less than 85% with respect to the total specific surface area. These mesopores and micropores can be measured using a specific surface area measuring instrument. As a specific measurement method, an absorption-desorption method based on a BET multipoint method is adopted.

The porous magnesia of substantially spherical shape having a silica layer obtained according to the present invention has an oil absorption of 300 to 600 ml/100 g, preferably 350 to 500 ml/100 g. This oil absorption can be measured by a Rub-out method using linseed oil etc.

For the porous magnesia of substantially spherical shape having a silica layer obtained according to the present invention, a sliding property (smooth and dry feel) as a feel of use for the skin is measured by a KES-SE friction feel measurement instrument (“KES-SE-DC Tester” from KATO TECH Co. Ltd). The porous magnesia has a friction coefficient (MIU) of not higher than 0.6, preferably 0.3 to 0.5. The method of measuring the MIU value is specifically described in JP, A, 2003-261796, and especially the method described in paragraph [0046] can be adopted.

The porous magnesia of substantially spherical shape having a silica layer obtained according to the present invention can find various uses as carriers for deodorants, antibacterials, catalysts, antidepressant and plastic additives, and body pigments. The porous magnesia of substantially spherical shape of the present invention can be used by preparing it into various forms/dosage forms especially as raw material for deodorants. That is, as desired, it can be processed into powder, granules, or pellets for use. For example, it can be used for various deodorants in a liquid state, a powder state, an emulsion, a lotion, a gel, a creamy state, a powder spray, a stick type, a foamed type, as well as an air sol form, a deodorizing sheet, etc., and for cosmetics having deodorization capabilities.

When used as the above described various deodorants and deodorant cosmetics, they can be used in combination with, for example, various kinds of oils, surfactants, germicides, vitamins, amino acids, anti-inflammatory agents, cold feeling imparting agents, etc. Such components include: oils and fats such as castor oil, sesame oil, soybean oil, and safflower oil; hydrocarbons such as bee wax, lanolin, and shellac; aliphatic acids such as succinic acid, tartaric acid, oleic acid, and citric acid; alcohols such as ethanol, isopropanol, cetanol, and oleyl alcohol; polyalcohols such as ethylene glycol, polyethylene glycol, and glycerin; sugars such as glucose, lactose, sorbitol, and xylitol; esters such as isopropyl adipate, lanolin acetate, and isopropyl myristate; soaps such as aluminum stearate, and magnesium stearate; soluble polymers such as gum Arabic, sodium alginate, carageenan, gelatin, and ethyl cellulose; non-ionic surfactants such as methylphenyl polysiloxane, and polyoxyethylene hardened castor oil; anionic surfactants such as alkylaryl sulfonates, and higher alkyl sulfate; preservatives such as alkyl para-oxybenzoate; vitamins such as vitamins A and D; hormones such as estradiol; organic coloring matters such as Red No. 2 and Blue No. 1; inorganic coloring materials such as mica, titanium, and zinc oxide; ultraviolet absorbing agents such as urocanic acid; and various propellants, purified water, antiperspirants such as aluminum hydroxychloride, microbicides, etc.

Hereinafter, the invention will be described in more detail based on examples.

### EMBODIMENTS

### Example 1

Six liters of deionized water are heated to 80° C. while stirring. Thereto, 5600 g of aqueous solution, in which 160 g of potassium sulfate, 40 g of sodium sulfate, and 1400 g of magnesium sulfate (MgSO₄.7H₂O) are dissolved into 4000 g of water, are simultaneously added dropwise keeping the pH at 9.5 using 15 wt % aqueous sodium carbonate solution. After completing the dropwise addition of these aqueous solutions, heating and stirring are stopped and the mixture is left standing for 16 hours. 10 liters of supernatant liquid are withdrawn therefrom and are added with 3 liters of water, and heated to 80° C. while stirring, and 1200 g of 5.6% aqueous sodium silicate solution are simultaneously added dropwise keeping the pH at 9.3 using dilute hydrochloric acid (1:2, that is concentrated hydrochloric acid is diluted with two times as large volume of water; hereinafter the same). After completing the dropwise addition, dilute hydrochloric acid (1:2) is further added dropwise thereto to obtain a suspension of pH 8.5. The suspension is filtered, washed with deionized water, dried at 110° C. and calcined at 550° C. to obtain porous magnesia of substantially spherical shape having a silica layer. A SEM observation shows that the resultant powder is substantially spherical particles having a structure in which thin platelets are combined or intersected in different directions and their mean particle diameter is 21 μm. Also an EDX observation confirms that silica is uniformly coated. The resultant substantially spherical porous magnesium has only mesopores. The oil absorption is 510 ml/100 g. Further, the friction coefficient measured by a KES-SE friction feel measurement instrument from KATO-Tech. Inc. is 0.39.

### Example-2

1.8 liters of deionized water are heated to 80° C. while stirring. Thereto, 1480 g of aqueous solution, in which 40 g of potassium alumosulfate (KAISO₄.9H₂O) and 240 g of magnesium sulfate (MgSO₄.7H₂O) are dissolved into 1200 g of water, are simultaneously added dropwise keeping the pH at 8.5 using an 15 wt % aqueous solution of sodium carbonate. After completing the dropwise addition of these aqueous solutions, heating and stirring are stopped and the mixture is left standing for 16 hours. 1.5 liters of supernatant liquid are withdrawn, then is heated to 80° C. while stirring, and 5.6% aqueous solution of sodium silicate is simultaneously added dropwise keeping the pH at 8.0 using dilute hydrochloric acid (1:2). After completing the dropwise addition, dilute hydrochloric acid (1:2) is further added dropwise making the pH of the suspension to be 6.5. The suspension is filtered, washed with deionized water, dried at 110° C., and calcined at 500° C. to obtain porous magnesia of substantially spherical shape complexed (doped) with aluminum having a silica layer. A SEM observation shows that the resultant powder is substantially spherical particles having a structure in which thin platelets are combined or intersected in different directions and their mean particle diameter is 40 μm. Also an EDX observation confirms that silica is uniformly coated. The resultant porous magnesia of substantially spherical shape has only mesopores. The oil absorption is 460 ml/100 g. The friction coefficient measured by KES-SE friction feel measurement instrument from KATO-Tech Inc. is 0.44.

### Example-3

1.8 liters of deionized water are heated to 85° C. while stirring. Thereto, 1406 g of aqueous solution, in which 40 g of potassium sulfate, 20 g of sodium sulfate, and 346 g of magnesium sulfate (MgSO₄.7H₂O) are dissolved into 1000 g of water, are simultaneously added dropwise keeping the pH at 9.2 using 20 wt % aqueous solution of sodium carbonate. After completing the dropwise addition, further 690 g of 2.2% aqueous sodium silicate solution are simultaneously added dropwise keeping the pH at 9.2 using dilute hydrochloric acid (1:2). After the dropwise addition, dilute hydrochloric acid (1:2) is further added dropwise to make the pH of the suspension to be 8.0. The suspension was filtered, washed with deionized water, dried at 110° C. and calcined at 500° C. to obtain porous magnesia of substantially spherical shape having a silica layer. A SEM observation shows that the resultant powder is substantially spherical particles having a structure in which thin platelets are combined or intersected in different directions and their mean particle diameter is 22 μm. Also an EDX observation confirms that silica is uniformly coated. The resultant porous magnesia of substantially spherical shape has only mesopores in the surface thereof. The oil absorption is 390 ml/100 g. The friction coefficient measured by KES-SE friction feel measurement instrument from KATO-Tech Inc. is 0.37.

### Example-4

Six liters of deionized water are heated to 80° C. while stirring. Thereto, 5600 g of aqueous solution, in which 160 g of potassium sulfate, 40 g of sodium sulfate, and 1400 g of magnesium sulfate (MgSO₄.7H₂O) are dissolved into 4000 g of water, are simultaneously added dropwise keeping the pH at 9.5 using 15 wt % aqueous sodium carbonate solution. After completing the dropwise addition of these aqueous solutions, heating and stirring are stopped and the mixture is left standing for 16 hours. 10 liters of supernatant liquid are withdrawn, added with 3 liters of water, and are heated to 80° C. while stirring, and 1200 g of 5.6% aqueous solution of sodium silicate are simultaneously added dropwise thereto keeping pH at 9.3 using dilute hydrochloric acid (1:2). After the dropwise addition, 415 g of aqueous solution, in which 11 g of potassium sulfate, 4 g of sodium sulfate, and 100 g of magnesium sulfate (MgSO₄.7H₂O) are dissolved into 300 g of water, are added dropwise simultaneously keeping the pH at 9.0 using 15 wt % aqueous sodium carbonate solution. Further, dilute hydrochloric acid (1:2) is added dropwise making the pH of the suspension to be 8.5.

The suspension is filtered, washed with deionized water, dried at 110° C. and calcined at 550° C. to obtain porous magnesia of substantially spherical shape having a silica layer. A SEM observation shows that the resultant powder is substantially spherical particles having a structure in which thin platelets are combined or intersected in different directions and their mean particle diameter is 21 μm. Also an EDX observation of the particles calcined after being coated with hydrated silicon oxide shows that silica is uniformly coated. The proportion of mesopores in the resultant porous magnesia of substantially spherical shape is 89.7%. The oil absorption is 480 ml/100 g. The friction coefficient measured by KES-SE friction feel measurement instrument from KATO-Tech Inc. is 0.42.

Measurement Method of Deodorization Rate (J. Soc. Cosmet. Chem. Japan 37(3) 202-209 (2003))

100 mg samples are taken from the examples in a 24 vial, and after spiking 30 μl of malodor component solution (isovaleric acid, torimethylamine, and 1-octene-3-one), are left standing for 5 minutes at 34° C. and thereafter subjected to a gas chromatography analysis in a PEG type column based on head-space GC-FID method. The peak area in a blank measurement (system without specimen) is also measured and thereby the reduction rate for each sample is calculated.

The results are shown in Table 1.

TABLE 1
Deodorization rate (%)

| Sample | isovaleric acid | trimethylamine | 1-octene-3-one |
|---|---|---|---|
| Example-1 | 98 | 85 | 77 |
| Example-2 | 73 | 95 | 78 |
| Example-3 | 99 | 44 | 34 |
| Example-4 | 99 | 90 | 62 |

The results in Table 1 confirm that the porous magnesia of substantially spherical shape having a silica layer according to the present invention exhibits extremely strong deodorization effects against 1-octane-3-one, which is a typical aging odor, as well as against isovaleric acid and trimethylamine which are foot odor and difficult to be deodorized, and the deodorization rates thereof are also very high.

Table 2 shows the composition and physical properties of the porous magnesia of substantially spherical shape having a silica layer according to the present invention.

TABLE 2
Physical properties of porous magnesia consisting of silica coated
substantially spherical particles

| | | | | | Mesopore | | |
|---|---|---|---|---|---|---|---|
| | | | Specific | Mesopore | specific surface | | |
| | | Particle | surface | specific | area/specific | Oil | |
| | Composition | size | area | surface area/ | surface area | absorption | |
| Example | (wt %) | (μm) | (m²/g) | (m²/g) | (%) | (ml/100 g) | MIU |
| Example-1 | 22 wt % SiO₂ | 21 | 76.6 | 76.8 | 100 | 510 | 0.39 |
| | 78 wt % MgO | | | | | | |
| Example-2 | 17 wt % SiO₂ | 40 | 71.8 | 71.6 | 100 | 460 | 0.44 |
| | 15 wt % Al₂O₃ | | | | | | |
| | 68 wt % MgO | | | | | | |
| Example-3 | 13 wt % SiO₂ | 22 | 51.5 | 51.5 | 100 | 390 | 0.37 |
| | 87 wt % MgO | | | | | | |
| Example-4 | 21 wt % SiO₂ | 21 | 77.8 | 69.8 | 89.7 | 480 | 0.42 |
| | 79 wt % MgO | | | | | | |

### INDUSTRIAL APPLICABILITY

As described so far, since the porous magnesia of the present invention exhibits a very strong deodorizing effect, and has a very large oil absorption and excellent usability for skin, it can be suitably used in cosmetics, especially deodorant cosmetics.

Moreover, since the porous magnesia of the present invention has a porous structure with a high proportion of mesopores, it can be used in application areas where such feature is required, such as carriers for antibacterials, catalysts, antidepressant and plastic additives, and body pigments.

1. Porous magnesia of substantially spherical shape comprising:
substantially spherical particles forming a base of said porous magnesia and having a structure in which thin platelets of a magnesium compound are combined and/or intersected in two or more different directions; and hydrated silicon oxide forming an outer layer of said particles.

2. The porous magnesia according to claim 1, further comprising a magnesium compound layer as the outer most layer.

3. The porous magnesia according to claim 1, wherein the amount of said hydrated silicon oxide is 5 to 50 wt % as SiO₂ with respect to the total weight of the porous magnesia.

4. The porous magnesia according to claim 1, wherein said magnesium compound is one or more kinds selected from the group consisting of hydrated oxide, basic carbonate, and oxide of magnesium, and said hydrated silicon oxide is hydrated silicon oxide and/or silica.

5. The porous magnesia according to claim 1, wherein said magnesium compound is a complex metal hydroxide, a complex metal carbonate, and/or a complex metal oxide between magnesium and one or more other metal components selected from the group consisting of aluminum, zinc, and iron.

6. The porous magnesia according to claim 5, wherein the atomic ratio of said other metal component to magnesium: M/Mg (where M is any of Al, Zn or Fe, or a mixture thereof) is not more than 0.95.

7. The porous magnesia according to claim 1, wherein the mean particle diameter of said porous magnesia is 5 to 50 μm.

8. The porous magnesia according to claim 1, wherein the proportion of the specific surface area of mesopores having a pore diameter of 2 to 50 nm is not less than 80% with respect to the total specific surface area of said porous magnesia.

9. The porous magnesia according to claim 1, wherein the oil absorption thereof is 300 to 600 ml/100 g.

10. The porous magnesia according to claim 1, wherein the friction coefficient thereof measured by a KES friction tester is not more than 0.6.

11. A preparation process of the porous magnesia according to claim 1, comprising the steps of:
simultaneously adding dropwise to water
(A-1) a single aqueous solution of magnesium metal salt or a mixed aqueous solution of magnesium metal salt and other metal salt, and
(B-1) an aqueous alkaline solution or an aqueous carbonate solution to obtain substantially spherical particles having a structure in which thin platelets composed of hydrated metal oxide and/or carbonate of those metals are combined and/or intersected in two or more different directions;
coating the surfaces of said particles with hydrated silicon oxide from
(B-2) an aqueous alkali metal silicate solution, and
(A-2) a dilute aqueous mineral acid solution; and separating, washing, drying and, if desired, calcining the resultant suspension.

12. The preparation process according to claim 11, comprising the further steps of:
onto the outer layer of the porous magnesia, further simultaneously adding dropwise
(A-3) a single aqueous solution of magnesium metal salt or a mixed aqueous solution of magnesium metal salt with other metal salt, and
(B-3) an aqueous alkaline solution or an aqueous carbonate solution to coat a hydrated oxide and/or carbonate of those metals; and separating, washing, drying and, if desired, calcining the resultant suspension.

13. The preparation process according to claim 11, wherein said single aqueous solution of magnesium metal salt or said mixed aqueous solution of magnesium metal salt with other metal salt includes sulfate ions, and the ion concentration ratio of sulfate ion/magnesium ion, or the ion concentration ratio of sulfate ion/magnesium ion plus other metal ion is 0.3 to 2.0.

14. A method of using the porous magnesia claim 1 comprising employing said porous magnesia as a carrier for an antibacterial, a catalyst, an antidepressant or a plastic additive, or a body pigment.

15. A method of using the porous magnesia claim 1 comprising employing said porous magnesia as a deodorant.

16. A deodorant containing the porous magnesia according to claim 1.

17. Deodorant cosmetics containing the porous magnesia according to claim 1.
